(19) United States
(12) Patent Application Publication
(10) Pub. No.: US 2021/0393181 A1
(43) Pub. Date: Dec. 23, 2021

(54) METHOD AND APPARATUS FOR MEASURING EMOTIONAL CONTAGION

(71) Applicant: SANGMYUNG UNIVERSITY INDUSTRY-ACADEMY COOPERATION FOUNDATION, Seoul (KR)

(72) Inventors: Sang In PARK, Seoul (KR); Min Cheol WHANG, Goyang-si (KR); Soo Ji CHOI, Sejong-si (KR); Dong Won LEE, Seongnam-si (KR); Sung Chul MUN, Seoul (KR)

(21) Appl. No.: 16/906,791

(22) Filed: Jun. 19, 2020

(51) Int. Cl. A61B 5/16; A61B 5/0456; A61B 5/00; G06F 3/01

(52) U.S. Cl. CPC A61B 5/165; A61B 5/0456; G06F 2203/011; A61B 5/7264; G06F 3/015; A61B 5/7246

(57) ABSTRACT

Provided is an emotional contagion evaluating method and apparatus using an electrocardiogram (ECG). The emotional contagion evaluating method includes: detecting ECG raw signals of two test participants involved in social interactions by using an ECG sensor; extracting ECG signals digitized by sampling the ECG raw signals at a certain sampling frequency; extracting R-peak to R-peak intervals (RRIs) from the ECG signals; extracting heart rhythm patterns (HRPs) from the RRIs; calculating a correlation coefficient (r) by using the HRPs of the two test participants; and evaluating emotional contagion between the two test participants by using the correlation coefficient (r).

### BACKGROUND

### 1. Field

One or more embodiments relate to a method and apparatus for measuring emotional contagion, and more particularly, to a method and apparatus for quantitatively evaluating the intensity of positive and negative emotional contagion.

### 2. Description of the Related Art

Emotions are exchanged through verbal and nonverbal information during social interaction. Dyadic or group interactions can influence one's mood, decision making, behavior, and even group-level dynamics. This process is known as emotional contagion. Emotional contagion is interchangeably called emotion transference and affective mimicry. During an interaction, a person will subconsciously mimic the other by synchronizing nonverbal behaviors such as facial expressions, gaze patterns, head movements, gestures, and others. The mood of a follower who mimics nonverbal behaviors is altered to approximate that of a leader who transfers the nonverbal behaviors. Past studies on emotional contagion have mostly focused on the effects of emotional contagion associated with individual and group performance. Change in members' mood in a group was obviously associated with task performance. Emotional contagion was greater when people felt happy and engaged in collective activity.

Despite the accumulated knowledge of the leader's effect on the follower and group dynamics, researchers have yet to examine how the leader is identified during social interactions. Past experiments have often involved a self-evaluating method to locate the direction and amount of emotion transference. The problem with the self-evaluating method is that the collected data solely depends on people's honesty and awareness of their own feelings. Additionally, the method was limited by the lack of quantitative evaluation of the direction and amount of emotion transference. Moreover, roles of the leader and the follower were kept constant and predetermined: teacher to student, performer to audience, worker to customer, and others. These interactions are restricted to one-way transfer, instead of interactions where every individual has an equal opportunity to be emotionally contagious to others. Identifying who transferred more emotion in person-to-person interactions or who transferred the most in group-dynamic interactions would not have been possible without the use of the self-evaluation method in a controlled setting.

### SUMMARY

One or more embodiments include a method and apparatus for measuring requirements of emotional contagion, direction of emotional contagion, and intensity between positive and negative contagion by using heart rhythm pattern (HRP) synchronization.

According to one or more embodiments, a method of measuring emotional contagion includes:

detecting ECG raw signals of two test participants involved in social interactions by using an ECG sensor;

extracting ECG signals digitized by sampling the ECG raw signals at a certain sampling frequency;

extracting R-peak to R-peak intervals (RRIs) from the ECG signals;

extracting heart rhythm patterns (HRPs) from the RRIs;

calculating a correlation coefficient (r) by using the HRPs of the two test participants; and

evaluating emotional contagion between the two test participants by using the correlation coefficient (r).

According to one or more embodiments, the RRIs may be extracted in a range of 500 ms to 1200 ms.

According to one or more embodiments, the evaluating of the emotional contagion between the two test participants may be performed with respect to negative emotion and positive emotion.

According to one or more embodiments, the two test participants may be classified into a leader and a follower, and synchronization of the follower's emotion with the leader's emotion may be evaluated.

According to one or more embodiments, the ECG raw signals may be measured by using Lead I of standard limb leads.

According to one or more embodiments, the correlation coefficient (r) may be obtained from a Pearson correlation coefficient (−1≤r≤1), and a positive correlation coefficient and a negative correlation coefficient may be compared with a correlation coefficient critical value to evaluate the emotional contagion between the test participants.

According to one or more embodiments, a correlation coefficient critical value to be compared with a positive correlation coefficient and a negative correlation coefficient may be 0.262 (±5%).

According to one or more embodiments, an emotional contagion evaluating apparatus using an electrocardiogram (ECG) for performing the above method includes:

the ECG sensor for detecting the ECG signals from the test participants;

a preprocessor for preprocessing the ECG signals; and

an analyzer for analyzing the emotion contagion between the two test participants by using signals obtained from the preprocessor.

According to one or more embodiments, the preprocessor may extract the RRIs in a range of 500 ms to 1200 ms.

According to one or more embodiments, the analyzer may perform the evaluating of the emotional contagion between the two test participants with respect to negative emotion and positive emotion.

According to one or more embodiments, the analyzer may classify the two test participants into a leader and a follower and may evaluate synchronization of the follower's emotion with the leader's emotion.

According to one or more embodiments, the analyzer may obtain the correlation coefficient (r) from a Pearson correlation coefficient (−1≤r≤1) and use a positive correlation coefficient and a negative correlation coefficient.

### BRIEF DESCRIPTION OF THE DRAWINGS

These and/or other aspects will become apparent and more readily appreciated from the following description of the embodiments, taken in conjunction with the accompanying drawings in which:

FIG. 1 shows an experimental environment according to one or more embodiments;

FIG. 2 shows an experimental procedure according to one or more embodiments;

FIGS. 3A through 3D show examples of a change or difference in a heart rhythm pattern (HRP) between an imitation task and a self-expression task, according to one or more embodiments;

FIG. 4 shows a comparison result of a correlation coefficient (r) of the HRP between a leader and a follower in an imitation task and a self-expression task, according to one or more embodiments;

FIGS. 5A through 5B show comparison examples of positively and negatively synchronized HRP according to one or more embodiments;

FIG. 6 shows a comparison result of a correlation coefficient of synchronized HRP and a leader's HRP, according to one or more embodiments;

FIG. 7 shows a comparison result of a correlation coefficient for emotional contagion between positive and negative, according to one or more embodiments;

FIG. 8 shows a rule base for evaluating positive emotional contagion, according to one or more embodiments;

FIG. 9 shows a verification result of the rule base of FIG. 8, according to one or more embodiments;

FIG. 10 shows a rule base for evaluating negative emotional contagion, according to one or more embodiments; and

FIG. 11 shows a verification result of the rule base of FIG. 8, according to one or more embodiments.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, a method and apparatus for evaluating emotional contagion according to one or more embodiments will be described in detail with reference to the accompanying drawings.

A heart rhythm pattern (HRP) has a significant correlation with an emotional state. The HRP has been found to vary depending on the emotional state of a person. The HRP of a frustrated person has been shown to have an irregular (negative) pattern, whereas an appreciative person has been shown to have a coherent (positive) pattern. Moreover, physiological synchrony is closely related to social relationships, group performance, and emotional contagion.

One or more embodiments include a method and apparatus for objectively and quantitatively recognizing emotional contagion through a pattern of heart responses according to a difference of emotional contagion caused during interaction.

According to one or more embodiments, raw data for evaluating a test participant's concentration is extracted by using an electrocardiogram (ECG) sensor, and the data is processed by a processing apparatus serving as an emotional contagion evaluating apparatus.

The processing apparatus or evaluating apparatus includes an image preprocessor and an analyzer, and the processing apparatus has an analysis tool or software and a hardware system for implementing the analysis tool or software. The processing apparatus may be a computer-based apparatus, a general-purpose computer including software having an algorithm and hardware capable of being driven by the software, or an exclusive-purpose apparatus.

A processing result obtained from the processing apparatus may be displayed by a display apparatus, and a general external interface device, for example, a keyboard, a mouse, etc., may be further included as an input means.

In detail, the emotional contagion evaluating apparatus may include: an ECG sensor for detecting an ECG signal from a test participant; a preprocessor for preprocessing the ECG signal; and an analyzer for analyzing emotional contagion between two test participants by using the signal obtained from the preprocessor.

According to one or more embodiments, the preprocessor may extract an RR interval (RRI) in a range of 500 ms to 1200 ms. The analyzer may evaluate emotional contagion between two test participants with respect to negative emotion and positive emotion. In addition, the analyzer may divide the two test participants into a leader and a follower and may evaluate synchronization of the follower's emotion with respect to the leader's emotion. Further, the analyzer may obtain the correlation coefficient from a Pearson correlation coefficient (r, −1 r≤≤1) to use a positive correlation coefficient and a negative correlation coefficient.

The following experiment was conducted to objectively evaluate an emotional contagion evaluating method and an apparatus using the emotional contagion evaluating method according to one or more embodiments.

<Participants>

64 undergraduate students (32 women), ranging in age from 20 to 29 years old (mean 25.2±4.3 years) participated in the experiment. All of the test participants had no family or personal history related to cardiovascular diseases and voluntarily participated in the experiment. Each test participant was paid $105.26. Written consent was obtained from each test participant prior to the experiment, and participants were required to abstain from alcohol, cigarettes, and caffeine for 12 hours prior to the experiment, and to sleep normally. All protocols used in this study were approved by the Institutional Review Board of Sangmyung University, Seoul, South Korea.

<Experimental Procedure>

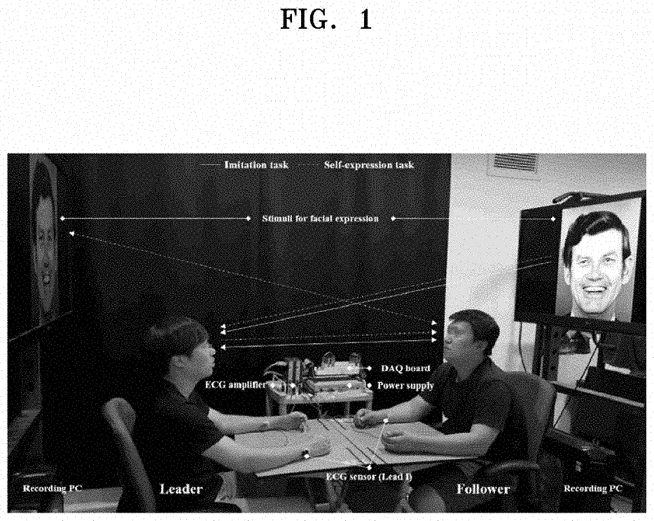

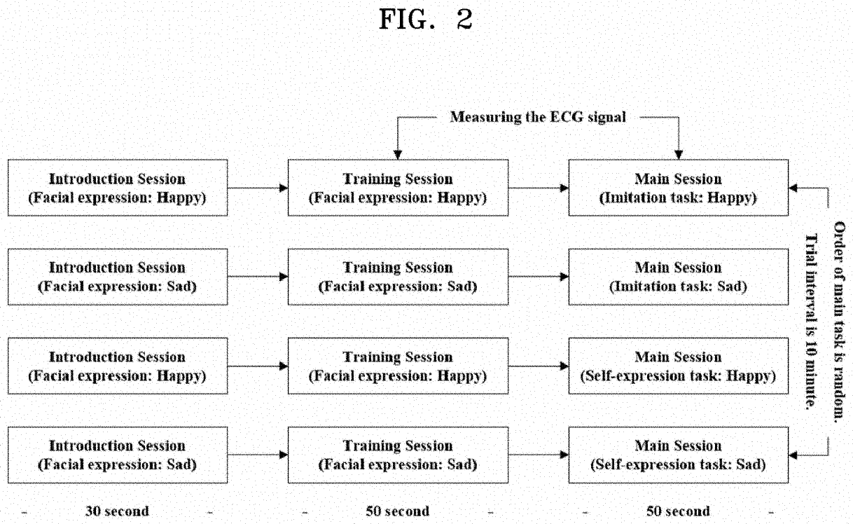

FIG. 1 shows an experimental environment according to one or more embodiments, and FIG. 2 shows an experimental procedure according to one or more embodiments.

Roles of a leader and a follower were randomly assigned to the test participants. As shown in FIG. 1, the leader and the follower were required to communicate with facial expressions of happiness and sadness presented through a display or face-to-face. The leader and the follower were seated face to face in comfortable chairs. Television (TV) displays presenting images of the facial expressions were behind them.

Referring to FIG. 2, an experimental task consisted of introduction, training, and main sessions. In the main session, an imitation task for mimicking the other party's facial expression and a self-expression task for making a facial expression oneself are performed. In the introduction session, the facial expressions of happiness or sadness defined by Ekman were explained for 30 seconds. The training session was to practice the facial expressions for 50 seconds. The main session was categorized into the imitation and self-expression tasks to cause emotional contagion. In the imitation task, the leader made a happy or sad face, following an expression presented through the display, and depicted the facial expression to the follower. The follower mimicked the leader's facial expression. In the self-expression task, the role of the leader was kept the same, but the follower mimicked a facial expression presented on the screen. Each task of the main session proceeded for 50 seconds.

All experiments consisted of four trials as below.

Trial 1: positive imitation (happiness)

Trial 2: negative imitation (sadness)

Trial 3: positive self-expression (happiness)

Trial 4: negative self-expression (sadness)

The above order of trials was random, and a time interval between trials was 10 minutes to minimize an effect of the previous stimulus. The facial expressions of happiness and sadness were based on facial expressions of Ekman's 6 basic emotions. During experimentation, electrocardiogram (ECG) signals of the leader and the follower were measured by using an ECG sensor.

<Data Acquisition, Signal Processing, and Analysis>

Data collection and processing were performed as below. An ECG was measured with the Lead-I method of standard limb leads. Raw signals or data obtained as such may be preprocessed by a preprocessor, and for example, an MP 100 power supply from Biopac System Inc. (USA), ECG 100C amplifiers, a NI-DAQ-Pad 9205 from National Instruments Inc. (USA), etc. may be used. Raw signals obtained from the sensor may be amplified through the preprocessor including these elements, and ECG signals digitized at a 500-Hz sampling frequency may be obtained.

In the ECG signals obtained through the preprocessing process, an R-peak was detected based on a QRS detection algorithm to calculate an R-peak to R-peak interval (RRI).

The RRI was calculated through signals having a normal interval in a range of 500 ms to 1200 ms. Beats per minute (BPM) used as a parameter for identifying the HRP was calculated through a multiplicative inverse of RRI. The signal processing was performed using the labVIEW 2015 (National Instruments Inc.). In this study, the presence of emotional contagion was defined by two tasks: emotional communication with another person (imitation task) or with a facial image on the screen (self-expression task).

A correlation coefficient (r) of the HRP between the leader and the follower was calculated in the training and main (imitation and self-expression tasks) sessions. A change of the correlation coefficient between the training and main sessions was compared in the imitation and self-expression tasks. Moreover, in this case, a difference of the correlation coefficient between positive and negative emotions was compared. Last, to confirm a direction of emotional contagion between the leader and the follower, synchronized HRP obtained by merging leaders' and followers' mean HRPs was calculated using data during the imitation task. A correlation coefficient with leader's and follower's HRPs was calculated during the training task.

<Statistical Analysis>

A difference of correlation between pre-task and post-task in emotional (non)contagion conditions was evaluated by an analysis of covariance (ANCOVA). One-way analysis of covariance compared dependent variables between groups, after the task, with a pre-task covariate. An independent t-test was used to test statistical significance of a difference of correlation between the leader and the follower, including positive and negative. In addition to the statistical significance, an effect size was calculated based on the eta-squared value (η²) and Cohen's d. In eta-squared (Cohen's d), standard values of 0.01 (0.20), 0.06 (0.50), and 0.14 (0.80) for the effect size are generally regarded as small, medium, and large, respectively. A Pearson correlation coefficient (r, −1≤r≤1) based on a normality test was used in the correlation analysis. A correlation coefficient approaching a value of −1 indicates a strong negative correlation, and that approaching a value of 1 indicates a strong positive correlation. All statistical analyses were performed through SPSS 17 (SPSS, Inc., Chicago, Ill.) software.

<Emotional Contagion and Non-Contagion>

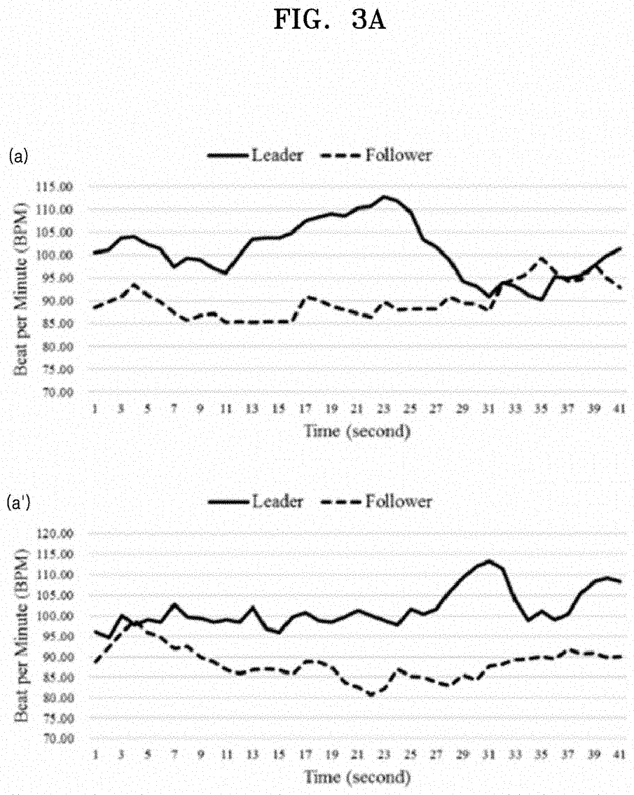

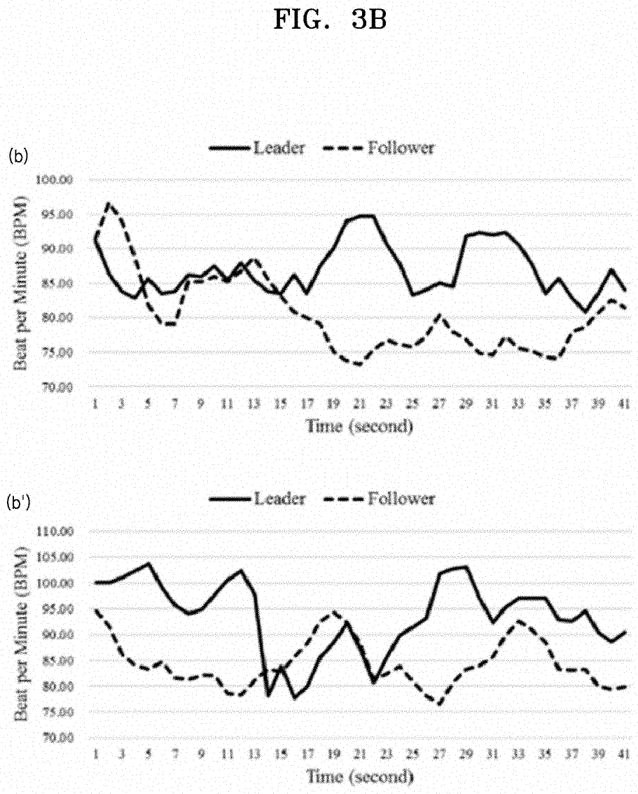

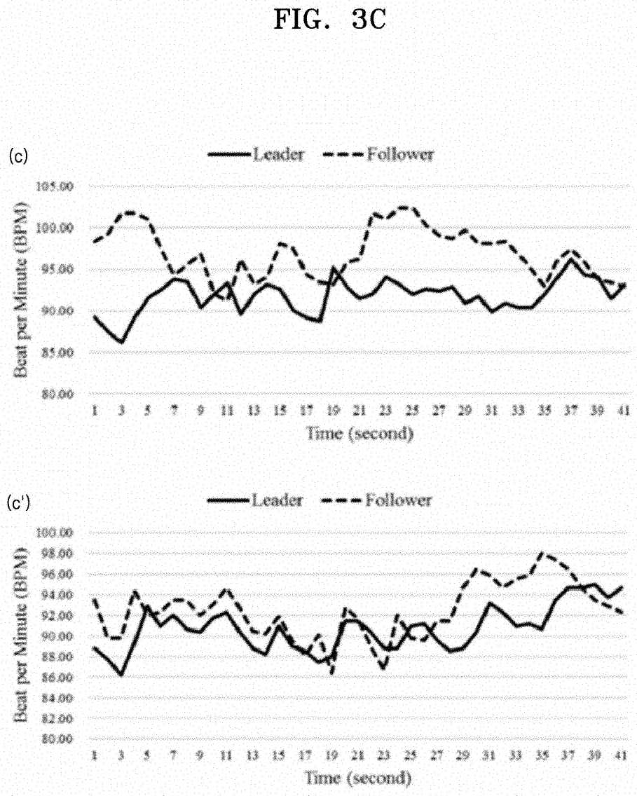

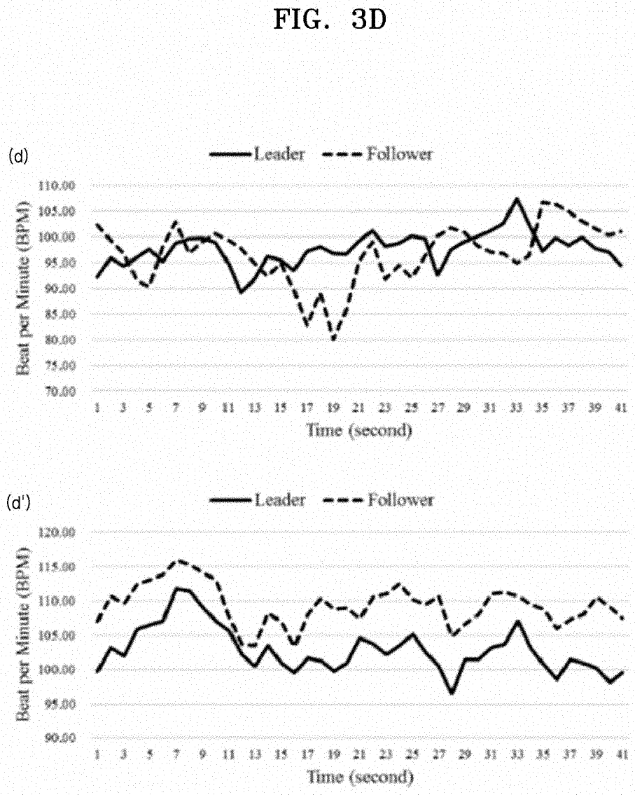

FIGS. 3A-3D show examples of a change or difference of the HRP between the imitation and self-expression tasks for test participant 7. In FIG. 3A, (a) shows a positive pre-self-expression task HRP, and (a′) shows a positive post-self-expression task HRP, In FIG. 3B, (b) shows a negative pre-self-expression task HRP, and (b′) shows a negative post-self-expression task HRP. In FIG. 3C, (c) shows a positive pre-imitation task HRP, and (c′) shows a positive post-imitation task HRP. In FIG. 3D, (d) shows a negative pre-imitation task HRP, and (d″) shows a negative post-imitation task HRP.

Before the task (emotional non-contagion condition), the correlation coefficient (r) between the leader and the follower in the pre-self-expression task of positive and negative was shown in −0.4436 and −0.3404, respectively. After the task (emotional contagion condition), the correlation coefficient was shown in −0.1424 and −0.0905, and there was no statistically significant difference. However, the correlation coefficient between the leader and the follower in the pre-imitation task of positive and negative (emotional non-contagion condition) was shown in 0.2455 and 0.0415, respectively. After the task (emotional contagion condition), the correlation coefficient was shown in 0.5518 and 0.7614 and significantly increased than before the task.

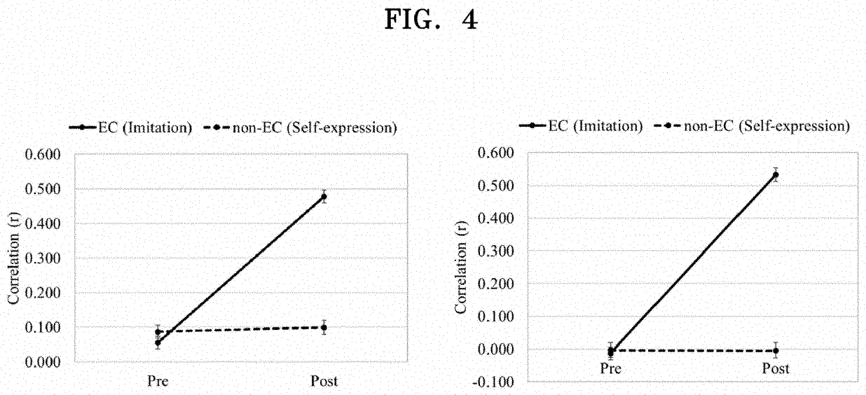

FIG. 4 shows a comparison result for a correlation coefficient of the HRP between the leader and the follower in imitation and self-expression tasks, and the left and right charts show the comparison result in positive and negative conditions, respectively.

Referring to FIG. 4, after the imitation and self-expression tasks, the correlation coefficient of the HRP between the leader and the follower significantly increased in the imitation task under both positive and negative emotion conditions.

Positive

F (1, 62)=195.609

p=0.000

η²=0.762

Negative

F (1, 62)=295.002

p=0.000

η²=0.829

However, before the imitation and self-expression tasks, the correlation coefficient of the HRP between the leader and the follower was not significant under either of the positive and negative emotion conditions.

Positive

F (1, 27)=2.709

p=0.105

η²=0.043

Negative

F (1, 27)=1.176

p=0.282

η²=0.019

The above test statistic F denotes a ratio of mean square regression (MSR) to mean square error (MSE).

Table 1 shows a detailed comparison result of the correlation coefficient (r) of the HRP between the leader and the follower in the imitation and self-expression tasks.

TABLE 1

| | | Emotional non-Contagion | | Emotional Contagion | | Synchronized | Synchronized |
|---|---|---|---|---|---|---|---|
| | | (Imitation Task) | | (Self-Expression Task) | | HRP with | HRP with |
| N = 32 | | Pre | Post | Pre | Past | Leader | Follower |
| Positive | Mean | 0.087 | 8.100 | 0.056 | 0.465 | 0.463 | 0.169 |
| | SD | 0.106 | 0.113 | 0.107 | 0.098 | 0.111 | 0.130 |
| | SE | 0.019 | 0.020 | 0.019 | 0.017 | 0.020 | 0.023 |
| Negative | Mean | −0.003 | −0.004 | −0.013 | 0.533 | 0.486 | 0.099 |
| | SD | 0.127 | 0.132 | 0.112 | 0.115 | 0.092 | 0.114 |
| | SE | 0.022 | 0.023 | 0.020 | 0.020 | 0.016 | 0.020 |

<Direction of Emotional Contagion>

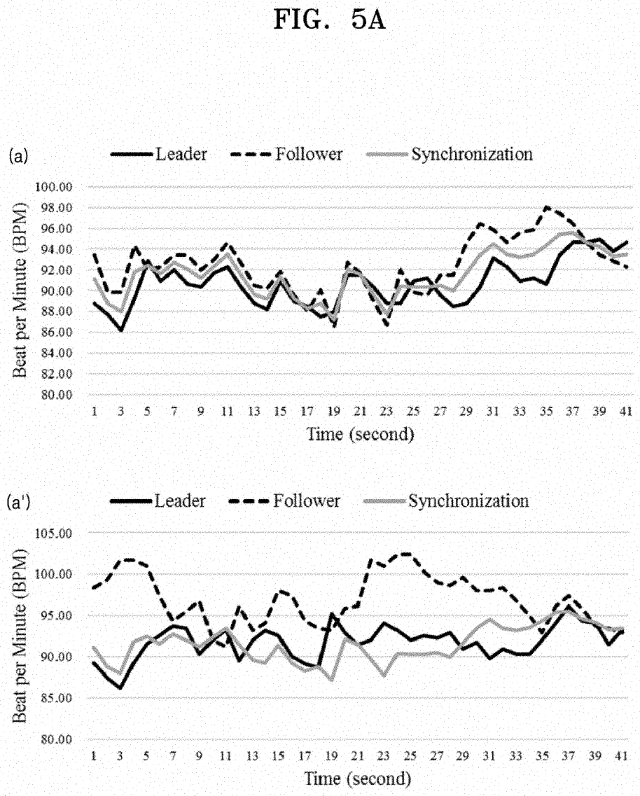

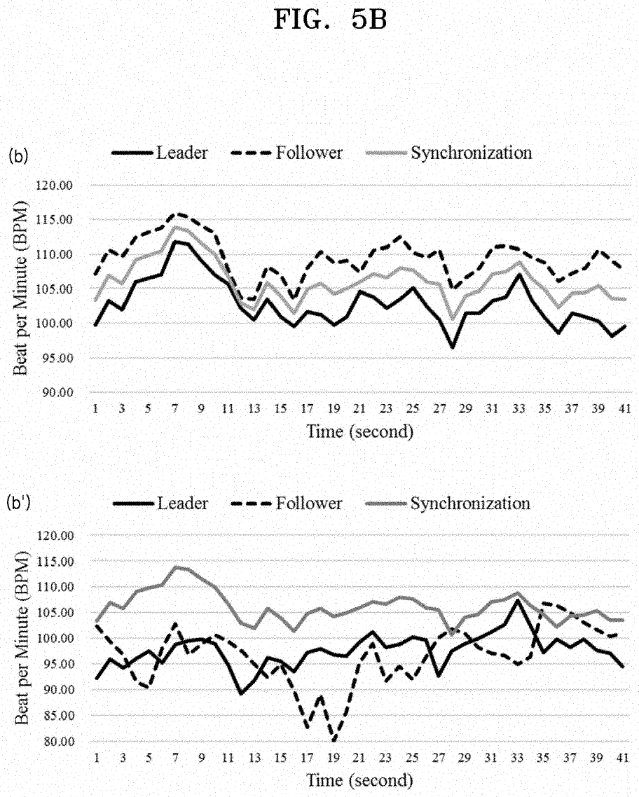

FIG. 5 shows a comparison example of synchronized HRP between the leader and the follower in emotional contagion for test participant 7, in which (A) shows the comparison of positively synchronized HRP, (B) shows the comparison of negatively synchronized HRP, (A′) shows the comparison of positively synchronized HRP with the leader and the follower, and (B′) shows the comparison of negatively synchronized HRP with the leader and the follower.

The synchronized HRP analyzed the correlation with the leader's and follower's HRPs before synchronization, a correlation coefficient of synchronized HRP and the leader's HRP before synchronization was 0.319, and a correlation coefficient of the follower's HRP was −0.265. Under a negative condition, a correlation coefficient of synchronized HRP and the leader's HRP before synchronization was 0.399, and a correlation coefficient of the follower's HRP before synchronization was −0.027.

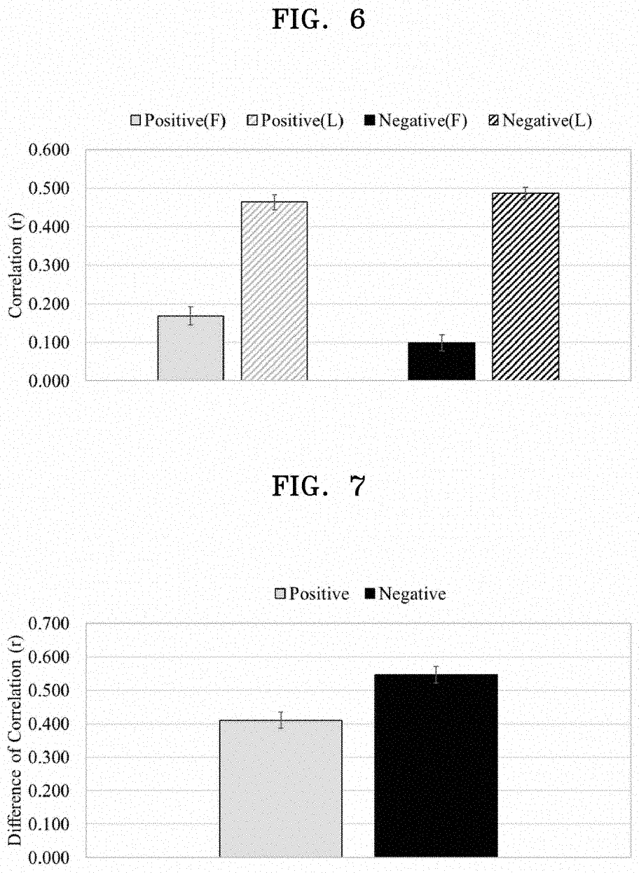

FIG. 6 shows a comparison result of a correlation coefficient of synchronized HRP and the leader's HRP.

In the statistical analysis, a correlation coefficient of synchronized HRP and the leader's HRP was significantly higher than that of synchronized HRP and the follower's HRP under both positive and negative conditions, as shown in FIG. 6.

Positive

t (62)=−9.589

p=0.000

Cohen's d=2.432

Negative

t (62)=−14.692

p=0.000

Cohen's d=0.627

<Emotional Contagion: Positive and Negative>

According to the examples of a change or difference between positive and negative for test participant 7 as described in the above and shown in FIGS. 3C, and 3D), the correlation coefficient after the task increased compared to before the task under both positive and negative conditions. However, the correlation coefficient of the negative condition (0.0415 to 0.7614) increased compared to that of the positive condition (0.2455 to 0.5518) after the emotional contagion task. As shown in FIG. 7, the correlation coefficient of the negative condition was significantly different from that of the positive condition (t (62)=−2.508, p=0.015, with a medium effect size (Cohen's d=3.736)). The detailed result is shown in Table 1.

According to one or more embodiments, the purpose of the study was to measure emotional contagion, determine a direction in expressive dyadic interactions, and identify a difference of positive and negative emotions with emotional contagion.

This study conducted an experiment for facial expressions of happiness and sadness between two persons causing emotional contagion. Emotional contagion and non-contagion were evaluated based on the imitation and self-expression tasks. The imitation task was to make facial expressions in a face-to-face situation, whereas the self-expression task involved self-expression based on facial expressions.

Overall, the present study yielded three important findings.

First, emotional contagion significantly increased a correlation coefficient of the HRP between two persons in both positive and negative emotions, but emotional non-contagion did not. The HRP has been reported to have a significant correlation with the emotional state, and synchronization of the HRP between two persons signifies that emotions between the leader and the follower were synchronized.

The previous studies reported that physiological synchrony was related to emotional contagion. Jaimovich's study showed that patterns of a musical performer's and a listener's GSR and HRV became similar. Moreover, a physiological link between two persons during conversational interaction was investigated with an electrodermal response (ED). An emotionally-arousing topic was measured between high-conflict and low conflict situations using 9-point Likert scale. Greater linkage was found in the ED among participants engaged in high-conflict situations compared to low-conflict or no-conflict situations. In this study, a correlation of the HRP between two persons was used to signify a degree of emotional synchrony, which could be measured emotionally.

Second, a direction of emotion transference was identified after the leader's and the follower's HRPs were synchronized. In this study, synchronized HRP was analyzed with an average value between the leader's and the follower's HRPs. A correlation coefficient between the leader's (before emotional contagion) and the synchronized HRP (after emotional contagion) was significantly higher than the follower's HRP. This phenomenon means that there was no significant difference between the leader's HRP before and after contagion although the follower's HRP before and after contagion changed significantly. Thus, the follower's HRP was shifted to match the leader's HRP through the emotional contagion activity. In this study, emotional contagion was defined by the synchronized HRP between the leader and the follower. The direction of emotional contagion was quantitatively detected by comparing the HRP before and after synchronization in each person.

Last, intensities of positive and negative emotional contagion were compared. During emotional contagion, a correlation coefficient of the negative emotion was significantly higher than that of the positive emotion. Increasing a correlation coefficient between two signals was shown to increase intensity of emotional contagion. Accordingly, this study found that an effect of transference for the negative emotion was higher than that for the positive emotion. In a study of emotional contagion investigated through strategic display of positive, negative, and neutral emotions, display of negative emotion was shown to have more effect on others than display of the positive emotion. Test participants responded to negative display of emotion with higher intensities, such as expressing more extreme demands, compared to positive display of emotion.

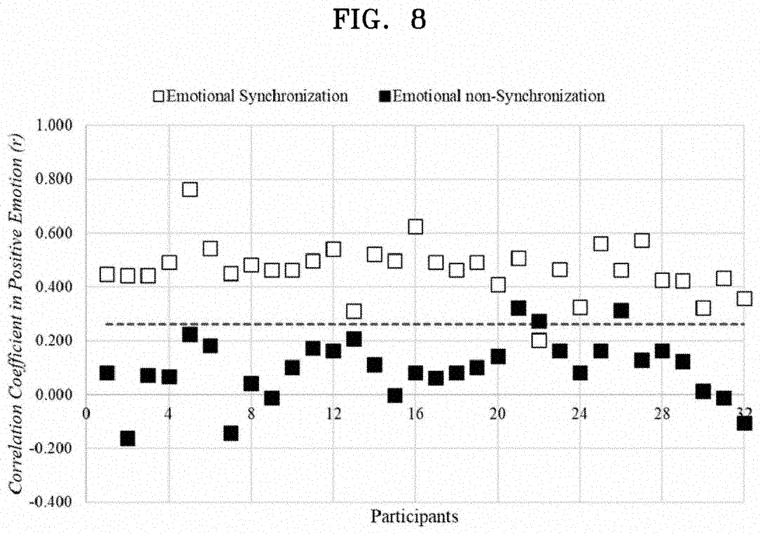

FIG. 8 shows a rule base for positive emotion and correlation coefficient distribution of test participants obtained during experimentation, according to one or more embodiments. A critical value of a correlation coefficient (r) determined according to an experiment of the present embodiment is 0.262. Referring to FIG. 8, the X-axis denotes test participants (each 32 samples, 64 people), and the Y-axis denotes a correlation coefficient of each sample. As shown in FIG. 8, correlation coefficients of the majority of test participants floating above a horizontal dash line of the correlation coefficient 0.262 (Y value) showed positive emotional contagion, whereas some negative results were shown near 0.262. In this regard, test participants whose correlation coefficients are greater than the correlation coefficient critical value 0.262 show positive emotional contagion, and correlation coefficients equal to or less than 0.262 show negative emotional contagion.

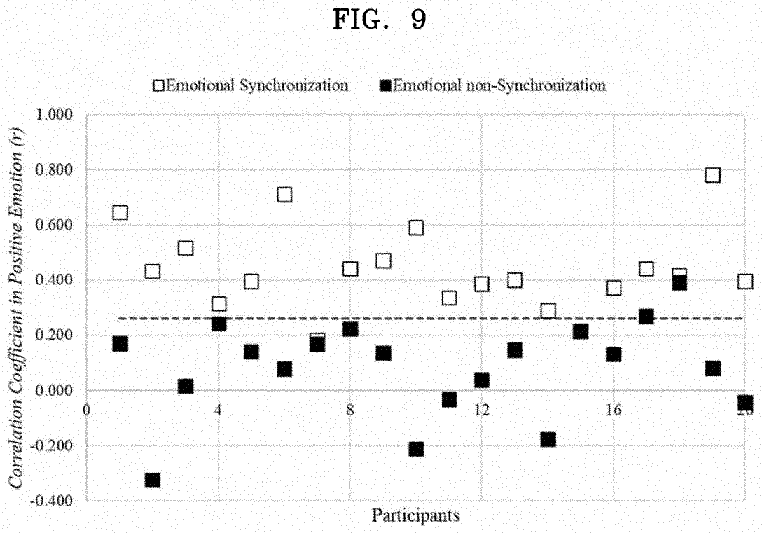

FIG. 9 shows a result of verifying the rule base shown in FIG. 8. A rule base of FIG. 9 is a result of verification with new data of each 20 samples, 40 people. As a result of verification with a critical value r=0.262 set as the rule base, accurate determination (accuracy 90%) of emotional contagion was verified in 36 samples from among 40 samples.

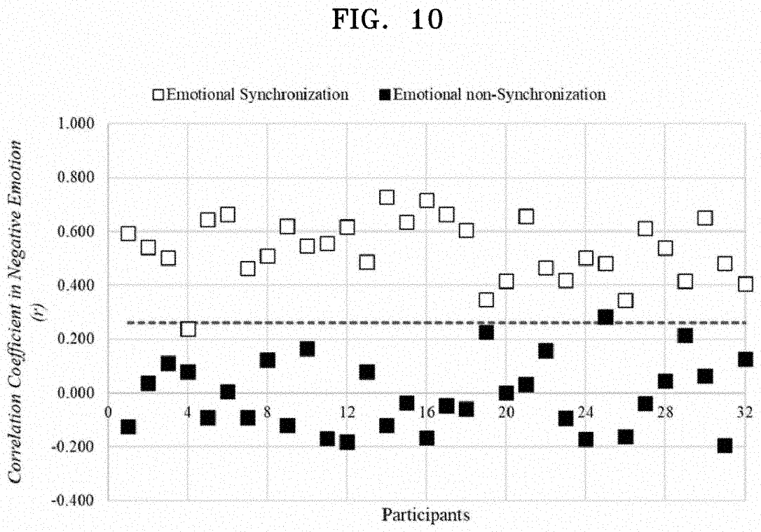

FIG. 10 shows a rule base for negative emotion and correlation coefficient distribution of test participants obtained during experimentation, according to one or more embodiments. A critical value of a correlation coefficient (r) for evaluating negative emotional contagion, determined according to an experiment of the present embodiment, is 0.262. Referring to FIG. 10, the X-axis denotes test participants (each 32 samples, 64 people), and the Y-axis denotes a correlation coefficient of each sample. As shown in FIG. 10, correlation coefficients of the majority of test participants floating above a horizontal dash line of the correlation coefficient 0.262 (Y value) showed negative emotional contagion, whereas few positive results were shown near 0.262. In this regard, test participants whose correlation coefficients are greater than the correlation coefficient critical value 0.262 show negative emotional contagion, and correlation coefficients equal to or less than 0.262 show positive emotional contagion.

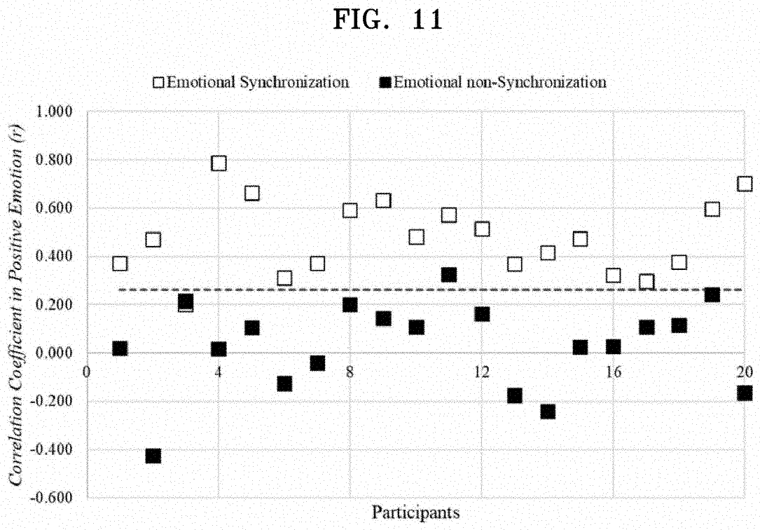

FIG. 11 shows a result of verifying the rule base shown in FIG. 10. A rule base of FIG. 11 is a result of verification with new data of each 20 samples, 40 people. As a result of verification with a critical value r=0.262 set as the rule base, accurate determination (accuracy 95%) of emotional contagion was verified in 38 samples from among 40 samples. Base on this result, a central value of critical values for evaluating positive emotion and negative emotion contagion may be 0.262, and an error thereof may be about 5%. Accordingly, a correlation coefficient critical value defined in one or more embodiments is 0.262, and an allowable error thereof may be defined as ±5%.

According to one or more embodiments, an emotional contagion evaluation method allows quantitative measurement of emotional contagion and its direction. To measure emotional contagion with identification of a leader may guide people to adjust their behaviors for improved emotion transference. A better understanding of emotional contagion and identification of a leader during interactions may improve the analysis of everyday social interactions such as work environments, social gatherings, and others.

The analysis of emotional contagion is crucial in gathering information of how our mind works during social interactions and may also be applied to numerous technologies and applications. For example, there are various training programs for people with social disorders, such as social skill training, facial emotion training, emotion recognition training, and others. In these training programs, patients may monitor whether their emotion was transferred and learn to behave accordingly. The identification of a leader may also be used to evaluate work settings. Taking into account emotions along with rationality has never been so important in business strategies. This physiological evaluation may help keep track of employees' leadership and persuasive skills, as well as customer service skills. Moreover, in an education domain, the present method may be applied to monitor engagement, empathy, class attitude and participation of students in online and offline domains. This study only verified two emotions, happiness and sadness. However, an emotional contagion measurement method according to one or more embodiments is highly scalable because heart rhythms reflect various emotional states.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. An emotional contagion evaluating method using an electrocardiogram (ECG), the method comprising: detecting ECG raw signals of two test participants involved in social interactions by using an ECG sensor;
extracting ECG signals digitized by sampling the ECG raw signals at a certain sampling frequency;
extracting R-peak to R-peak intervals (RRIs) from the ECG signals;
extracting heart rhythm patterns (HRPs) from the RRIs;
calculating a correlation coefficient (r) by using the HRPs of the two test participants; and
evaluating emotional contagion between the two test participants by using the correlation coefficient (r).

2. The method of claim 1, wherein
the RRIs are extracted in a range of 500 ms to 1200 ms.

3. The method of claim 1, wherein
the evaluating of the emotional contagion between the two test participants is performed with respect to negative emotion and positive emotion.

4. The method of claim 1, wherein
the two test participants are classified into a leader and a follower, and synchronization of the follower's emotion with the leader's emotion is evaluated.

5. The method of claim 1, wherein
the ECG raw signals are measured by using Lead I of standard limb leads.

6. The method of claim 1, wherein
the correlation coefficient (r) is obtained from a Pearson correlation coefficient (−1≤r≤1), and a positive correlation coefficient and a negative correlation coefficient are compared with a critical value to evaluate the emotional contagion between the test participants.

7. The method of claim 2, wherein
the correlation coefficient (r) is obtained from a Pearson correlation coefficient (−1≤r≤1), and a positive correlation coefficient and a negative correlation coefficient are compared with a critical value to evaluate the emotional contagion between the test participants.

8. The method of claim 3, wherein
the correlation coefficient (r) is obtained from a Pearson correlation coefficient (−1≤r≤1), and a positive correlation coefficient and a negative correlation coefficient are compared with a critical value to evaluate the emotional contagion between the test participants.

9. The method of claim 4, wherein
the correlation coefficient (r) is obtained from a Pearson correlation coefficient (−1≤r≤1), and a positive correlation coefficient and a negative correlation coefficient are compared with a critical value to evaluate the emotional contagion between the test participants.

10. The method of claim 5, wherein
the correlation coefficient (r) is obtained from a Pearson correlation coefficient (−1≤r≤1), and a positive correlation coefficient and a negative correlation coefficient are compared with a critical value to evaluate the emotional contagion between the test participants.

11. The method of claim 6, wherein
a correlation coefficient critical value to be compared with a positive correlation coefficient and a negative correlation coefficient is 0.262 (±5%).

12. An emotional contagion evaluating apparatus for performing the method of claim 1, the apparatus comprising:
the ECG sensor for detecting the ECG signals from the test participants;
a preprocessor for preprocessing the ECG signals; and
an analyzer for analyzing the emotion contagion between the two test participants by using signals obtained from the preprocessor.

13. The apparatus of claim 12, wherein
the preprocessor extracts the RRIs in a range of 500 ms to 1200 ms.

14. The apparatus of claim 12, wherein
the analyzer performs the evaluating of the emotional contagion between the two test participants with respect to negative emotion and positive emotion.

15. The apparatus of claim 12, wherein
the analyzer classifies the two test participants into a leader and a follower and evaluates synchronization of the follower's emotion with the leader's emotion.

16. The apparatus of claim 12, wherein
the correlation coefficient (r) is obtained from a Pearson correlation coefficient (−1≤r≤1), and a positive correlation coefficient and a negative correlation coefficient are used to evaluate the emotional contagion between the test participants.

17. The apparatus of claim 13, wherein
the correlation coefficient (r) is obtained from a Pearson correlation coefficient (−1≤r≤1), and a positive correlation coefficient and a negative correlation coefficient are used to evaluate the emotional contagion between the test participants.

18. The apparatus of claim 14, wherein
the correlation coefficient (r) is obtained from a Pearson correlation coefficient (−1≤r≤1), and a positive correlation coefficient and a negative correlation coefficient are used to evaluate the emotional contagion between the test participants.

19. The apparatus of claim 15, wherein
the correlation coefficient (r) is obtained from a Pearson correlation coefficient (−1≤r≤1), and a positive correlation coefficient and a negative correlation coefficient are used to evaluate the emotional contagion between the test participants.

20. The apparatus of claim 16, wherein
a correlation coefficient critical value to be compared with a positive correlation coefficient and a negative correlation coefficient is 0.262 (±5%).
